(19) United States
(12) Patent Application Publication
(10) Pub. No.: US 2019/0074076 A1
(43) Pub. Date: Mar. 7, 2019

(54) HEALTH HABIT MANAGEMENT

(71) Applicant: KONINKLIJKE PHILIPS N.V., EINDHOVEN (NL)

(72) Inventors: AKI SAKARI HARMA, EINDHOVEN (NL); JAN MARTIJN KRANS, DEN BOSCH (NL); DIETWIG JOS CLEMENT LOWET, EINDHOVEN (NL); SASKIA VAN DANTZIG, UTRECHT (NL)

(21) Appl. No.: 15/549,253

(22) PCT Filed: Feb. 17, 2016
(86) PCT No.: PCT/IB2016/050853

§ 371 Date: Aug. 7, 2017

Related U.S. Application Data
(60) Provisional application No. 62/119,879, filed on Feb. 24, 2015.

(51) Int. Cl. G16H 20/00; G16H 10/60; G16H 50/70; G16H 80/00

(52) U.S. Cl. CPC G16H 20/00; G16H 80/00; G16H 50/70; G16H 10/60

(57) ABSTRACT

Methods and systems for managing a user's health habits. Various embodiments of the invention allow the user to register habits to a habit registry. Relevant sensor and other data is collected contemporaneously with the registration of the behavior and is stored in association with the registered habit. In normal use, a configured processor attempts to detect occurrences of a previously registered habit by comparing subsequently collected data to previously stored data. When collected and stored data match, a registered health habit is detected, the occurrence may be added to a health habit log, and the feedback may be provided to the user. The feedback assists the user to achieve his goals in health habit management.

### TECHNICAL FIELD

The present invention relates to methods and apparatus for the management of health habits, and in particular to methods and apparatus for registering a user's health habits and providing feedback to the user to achieve health habit goals.

### BACKGROUND

For an individual concerned with effective health management, it is important to develop positive habits, i.e., activities that are regularly performed that help the user achieve a healthful state, or suppress negative habits. Various devices are known that permit a user to track their activities, including devices that track the position of the user (e.g., GPS) and devices that track the movement of the user based on accelerometers and other sensors.

There are several problems with these current approaches. It is possible to find recurring patterns in multidimensional sensor data, but it is difficult to determine when those patterns actually represent meaningful habits or behaviors of a user. Even if those patterns can be recognized as habits or behaviors, it can be difficult to offer feedback on those behaviors that are appropriate to the user's context. For example, a system that recommends that a person walk more may be ineffective if the person lives in an area where walks are neither safe nor convenient.

Accordingly, there is a need for methods and apparatus that recognize health related behavior in a reliable way and provide behavioral feedback that fits the context of the user, encouraging him to develop and follow new healthy behaviors.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Generally speaking, the present invention relates to methods and systems for managing a user's health habits. Various embodiments of the invention allow the user to register positive or negative habits to a habit registry such that they can be later detected from sensor data. Feedback is provided to the user that assists the user to achieve his goals in habit management.

For example, a user may register at a computing unit a specific behavior as a positive or negative habit during or soon after the execution of the behavior. A data collection device receives relevant sensor and other data that is available contemporaneously with (i.e., in a particular time period before, during, and/or after the registration of the behavior) and stores the data in association with the registered habit. During normal use, a configured processor attempts to detect subsequent occurrences of a registered habit by comparing subsequent data to the stored data. When a match between stored and subsequent data is detected, the registered habit is identified, the occurrence may be added to a habit log, and the system may provide feedback to the user concerning the habit.

In one aspect, embodiments of the present invention relate to a method for managing a user's health habits with a computing unit. The method includes receiving user data at the computing device from at least one data source, receiving a registration of a health habit at an interface, and associating the user data with the health habit in response to the receipt of the registration of the health habit.

In one embodiment, the method includes identifying a registered health habit from the received user data by matching the received user data with the user data associated with the registered health habit. In one embodiment, identifying a registered health habit includes computing a cross-correlation between the received user data and the user data associated with the registered health habit. In one embodiment, the method includes providing feedback to the user based on the identified health habit. In one embodiment, the feedback concerns at least one of the user data associated with the health habit, received user data, and differences between the user data associated with the health habit and the received user data.

In one embodiment, the user data is selected from the group consisting of the user's calendar data, the user's communication data, the user's vital signs data, the user's motion data, the user's position data, the user's electronic transaction data, the user's height above sea level, the identity of specific persons nearby the user, and the user's weather data. In one embodiment, the at least one data source is selected from the group consisting of an accelerometer, an audio sensor, a video sensor, a location sensor, a movement sensor, an orientation sensor, a skin conductance sensor, a respiration sensor, a glucose level sensor, and a heart rate sensor.

In one embodiment, the method includes changing the power state of the data source in response to matching the received user data with the user data associated with the registered health habit. In one embodiment, the health habit is suggested by the computing unit prior to registration.

In another aspect, embodiments of the present invention relate to a system for managing a user's health habits. The system includes a processor, at least one data source, and computer executable instructions operative on the processor for receiving user data from at least one data source, receiving a registration of a health habit at an interface, and associating the user data with the health habit in response to the receipt of the registration of a health habit.

In one embodiment, the system includes computer executable instructions operative on the processor for identifying a registered health habit from the received user data by matching the received user data with the user data associated with the registered health habit. In one embodiment, the computer executable instructions operative on the processor for identifying a registered health habit include computer executable instructions operative on the processor for computing a cross-correlation between the received user data and the user data associated with the registered health habit.

In one embodiment, the system includes computer executable instructions operative on the processor for providing feedback to the user based on the identified health habit. In one embodiment, the feedback concerns at least one of the user data associated with the health habit, received user data, and differences between the user data associated with the health habit and the received user data.

In one embodiment, the user data is selected from the group consisting of the user's calendar data, the user's communication data, the user's vital signs data, the user's motion data, the user's position data, the user's electronic transaction data, and the user's weather data. In one embodiment, the at least one data source is selected from the group consisting of an accelerometer, an audio sensor, a video sensor, a location sensor, a movement sensor, an orientation sensor, and a heart rate sensor.

In one embodiment, the system includes computer executable instructions operative on a processor for changing the power state of the data source. These changes are performed in response to matching received user data with the user data associated with the registered health habit. In one embodiment, the system includes computer executable instructions operative on said processor for suggesting the health habit that is subsequently registered.

In another aspect, embodiments of the present invention relate to a system for managing a user's health habits with a computing unit. The system includes means for receiving user data from at least one data source, means for receiving a registration of a health habit at an interface, and means for associating the user data with the health habit in response to the receipt of the registration of a health habit.

In one embodiment, the system includes means for identifying a registered health habit from the received user data by matching the received user data with the user data associated with the registered health habit. In one embodiment, the means for identifying a registered health habit includes means for computing a cross-correlation between the received user data and the user data associated with the registered health habit.

In one embodiment, the system includes means for providing feedback to the user based on the identified health habit. In one embodiment, the feedback concerns at least one of the user data associated with the health habit, received user data, and differences between the user data associated with the health habit and the received user data.

In one embodiment, the user data is selected from the group consisting of the user's calendar data, the user's communication data, the user's vital signs data, the user's motion data, the user's position data, the user's electronic transaction data, and the user's weather data. In one embodiment, the at least one data source is selected from the group consisting of an accelerometer, an audio sensor, a video sensor, a location sensor, a movement sensor, an orientation sensor, and a heart rate sensor.

In one embodiment, the system includes means for changing the power state of the data source in response to matching the received user data with the user data associated with the registered health habit. In one embodiment, the system includes means for suggesting the health habit that is subsequently registered.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF DRAWINGS

Non-limiting and non-exhaustive embodiments are described with reference to the following Figures in which:

FIG. 1 is a flowchart illustrating one embodiment of a method for health habit management in accord with the present invention; and

FIG. 2 is a block diagram presenting one embodiment of a system for health habit management in accord with the present invention.

In the drawings, like reference characters generally refer to corresponding parts throughout the different views. The drawings are not necessarily to scale, emphasis instead being placed on the principles and concepts of operation.

### DETAILED DESCRIPTION

Various embodiments are described more fully below with reference to the accompanying drawings, which form a part hereof, and which show specific exemplary embodiments. However, embodiments may be implemented in many different forms and should not be construed as limited to the embodiments set forth herein; rather, these embodiments are provided so that this disclosure will be thorough and complete, and will fully convey the scope of the embodiments to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one embodiment of the invention. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are the means used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the present invention include process steps and instructions that could be embodied in software, firmware or hardware, and when embodied in software, could be downloaded to reside on and be operated from different platforms used by a variety of operating systems.

The present invention also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein, and any references below to specific languages are provided for disclosure of enablement and best mode of the present invention.

In addition, the language used in the specification has been principally selected for readability and instructional purposes and may not have been selected to delineate or circumscribe the inventive subject matter. Accordingly, the disclosure of the present invention is intended to be illustrative, but not limiting, of the scope of the invention, which is set forth in the claims.

### Introduction

Embodiments of the present invention relate to methods and systems for managing a user's health habits. Some aspects of the invention include a processor, at least one data source, and computer executable instructions for receiving user data from the at least one data source, receiving a registration of a health habit at an interface, and associating the user data with the health habit in response to the receipt of the registration of a health habit. These embodiments allow a user to register positive or negative habits in a health habit registry such that they can be later detected from subsequent sensor data. In one embodiment of the invention, the registered health habits comprise activities which increase the energy consumption of the body, and which can be measured by an increase in the physical activity of the user as evidenced by, e.g., heart rate, skin temperature, skin perspiration, etc. The user may register a health habit using a graphical user interface by selecting potential times, places, friends, devices, circumstances for the execution of the habit.

One example of a “health habit” is using the stairs instead of the escalator in a subway station. Taking the stairs leads to an increase in the heart rate of the user as well as an increase in the user's energy expenditure when compared to the case where the user takes the escalator. The “context” for this health habit includes the time of day when the user is commuting to work or back and the location of the user at a subway station. The context can be measured or determined using, e.g., a clock and some localization means based on global or local positioning systems.

Some health habits are performed using specific devices (e.g., a TV, tablet, refrigerator, toothbrush, etc.). Information from these devices could also be used to provide context for a health habit, limiting the monitoring for future occurrences of a registered habit to times when the device is in use.

Similarly, some habits may only be performed in the presence of specific persons, and the state of certain sensors might be switched on or off when these persons are nearby. For example, in one embodiment, heart rate sensing is based on a photoplethysmograph (PPG) integrated in a wrist-worn device. When a measured context matches the context of a previously registered habit, the PPG sensor electronics is switched on to record possible changes in the heart rate that indicate the execution of the health habit related to the context. Otherwise, the PPG may be left disabled to conserve power.

During normal use, the configured processor attempts to detect subsequent occurrences of a registered habit by comparing contemporaneous data with the stored data. When a registered habit is detected, the occurrence may be added to a habit log and the system may provide feedback to the user depending on the type of the habit, thus assisting the user to achieve his goals for health habit management.

### Embodiments

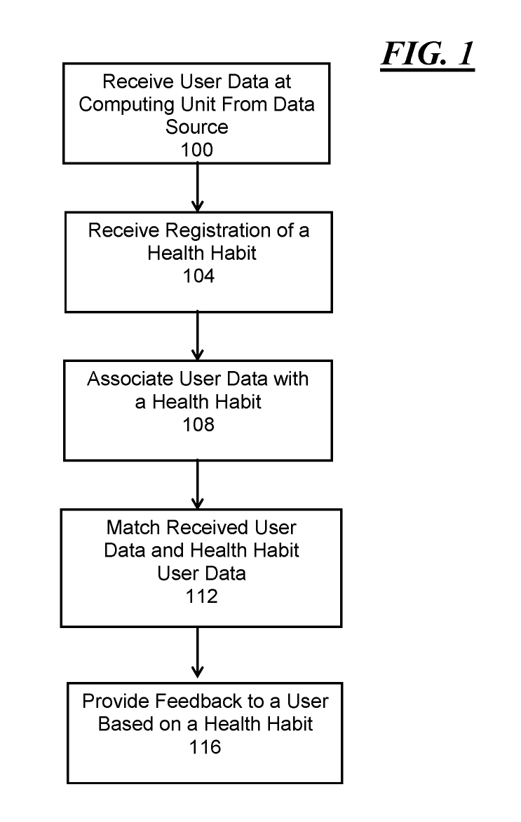

FIG. 1 is a flowchart illustrating one embodiment of a method for managing a user's health habits in accord with the present invention. A data collection device receives sensor and other relevant data from at least one data source, e.g., a sensor, that is contemporaneous with the performance of a particular health habit (i.e., occurring before, during, and after the performance of the habit) and stores the data in a persistent storage (e.g., non-volatile storage, etc.) (Step 100).

In various embodiments, the received data may comprise the user's calendar data, the user's communication data, the user's vital signs data, the user's motion data, the user's position data, the user's electronic transaction data, the user's height above sea level, the identity of specific persons nearby the user, the user's weather data, etc. In various embodiments, the data source may be an accelerometer, an audio sensor, a video sensor, a location sensor, a movement sensor, an orientation sensor, a skin conductance sensor, a respiration sensor, a glucose level sensor, or a heart rate sensor.

In operation, a user registers a new health habit at an interface (Step 104). In some embodiments of the invention, this can involve pressing a button on a mobile computing device to register a good health habit or a bad health habit. In other embodiments, the mobile computing device has a touch sensitive screen that permits a user to register a health habit.

In other embodiments, the user may also provide information as to whether a registered habit is related to weight management, physical activity, or tobacco use, for example. The habit registration interface may comprise a speech interface or any other input device.

In some embodiments, health habit registrations are stored in an activity log which may be accessible to the user later. The system may also provide alerts to the user in the event that a habit was not detected (e.g., an expected change in the heart rate or activity level in the registered context did not occur).

In some embodiments the system may propose one or more habits which the user can select and register. The proposed habits may be specific (e.g., “use stairs at the subway station on the way back from work”) or generally indicate a potential context for a new habit (e.g., “you could be more active on Monday afternoons”). In the latter case the registration of a habit means in practice that a user registers a context of a behavior change but the actual execution of the habit may remain unknown to the sensing system.

When the user registers a habit, the system stores the collected data that is roughly contemporaneous with the registration (i.e., collected data somewhat preceding the registration, collected data coinciding with the registration, and collected data somewhat following the registration) (Step 108). The collection period may be of a fixed duration, and the duration can be selected automatically based on the collected data. The duration may also be chosen by the user. For example, the stored data may include location data, time of day, activity data and, for example, measurements related to heart rate or other physiological measures.

For example, after eating a chocolate bar, the user may register that “what I just did was a bad habit and I want to stop it,” or after a 15 minute walk a user may indicate “the last 15 minutes was an example of a good habit I want to enforce which I will refer to as ‘evening walk’.” In response, the data collection device collects and stores the sensor data corresponding to, the last minute of activity, or the last 15 minutes of activity, continuing these two examples.

A registered health habit is then identified by matching subsequently received user data with the user data associated with the registered health habit(s) (Step 112). In one embodiment, a registered health habit may be identified by computing a cross-correlation between the subsequently received user data and the user data associated with the registered health habit(s). For example, a sliding normalized cross-correlation may be computed between the currently-captured (i.e., the subsequent) data and the user data previously stored in the registry in connection with the health habit(s). When the normalized (i.e., Pearson) correlation is above a threshold value (such as 0.9), a recurrence of a registered health habit is identified.

In other embodiments of the invention, a registered health habit may be identified by use of template matching, i.e., computing a similarity metric between each set of previously registered health habit data and subsequent incoming data. The similarity measure may be, for example, the Pearson correlation coefficient between the incoming data and the previously-registered sets of data or an Euclidean distance measure between the corresponding data points. The similarity measure may also be based on comparison of parametric or non-parametric representations of distribution parameters representing the statistics of the measures. A typical similarity measure to compare distribution parameters is the Kullback-Leibler divergence measure. In the case of missing data values the similarity measure can be computed between the data elements that are available or the computation of the similarity may contain the estimation of the missing values based on a generic data model that describes the dependencies between different measures. The matching process may also involve normalizations and non-linear manipulations of the subsequent incoming data and/or the previously-registered health habit data packets, and the computation of descriptive features from the data, before the computation of a similarity metric.

In another aspect, the identification of a registered health habit can be used to control the power state of one or more user data sources. For example, if one of the registered health habits concerns running, and subsequent data indicates that the user is running, then power can be supplied to, e.g., an accelerometer or position sensor, to enable the tracking of the user's route. Conversely, when subsequent data does not match a registered habit, power can be removed from one or more user data sources, thereby reducing power consumption and extending battery life in a portable device.

In yet another aspect of the present invention, the subsequently-captured sensor data may be organized into semantically meaningful segments representing different contexts of the user. Several computations and health-related measurements are then performed in particular context segments. Based on the measurements and segmentation, the system generates a number of context claims that characterize the properties of one context or relative differences between two or more contexts (Step 112).

In one embodiment a context is a combination of a place and a time. The place may be a specific physical location (e.g., identified by latitude and longitude coordinates), or a semantic location such as a workplace or a shop. The time may be, for example, a time of a day, a particular weekday, a particular day of the month, or a holiday. A context may also be characterized by the movement of the user, for example, on the way to work, or travelling somewhere. In other embodiments context may include weather condition and nearby persons.

Embodiments of the invention collect measurements for each identified recurring context of the user. In one embodiment a context claim is a statement of the following form: “Measurement 1” is “lower/higher in Context A than in Context B.” For example, a context claim could be as follows: “Your average heart rate on the way to work is typically higher (95 bpm) than your average heart rate on the way to home (82 bpm).” In addition to a textual representation it is also possible to present the context claim to the user in various alternative forms such as a graphic illustration or spoken announcement. The statement may also highlight a context of a maximum or a minimum of a measure, for example: “your maximum heart rate in a week is typically reached on Tuesdays on the way to work”.

The context claims are presented to the user and the user is encouraged to consider opportunities to change the behavior reflected in the context claims (Step 116). If the user identifies such an opportunity he may then register a new habit that changes the behavior (Step 104). The system may then monitor the changes in the context claim over time and provide additional feedback to the user to reinforce the development of healthier habits (Step 116).

In another aspect of the invention, the invention provides feedback to the user based on previously registered health habit(s) (Step 116). In some embodiments, the feedback concerns at least one of the user data associated with the health habit, subsequently received user data, and differences between the user data associated with the health habit and subsequently received user data (Step 116).

In yet another aspect of the invention, the system provides feedback to the user based on a user's context (Step 116). For example, on Monday morning the user may be presented with feedback that compares the user's current Monday with a typical Monday morning of the user, to other days, to past Mondays, to an average Monday. The user may also be presented with feedback that does not necessarily involve a direct comparison, for example, feedback related to the time-of-the-year, the weather, or the location of the user (Step 116).

In various embodiments the content of particular items of feedback is formed by comparative statements based on: (a) time-based statistics such as summaries of weeks, months, weekdays, etc; (b) location-based statistics such as the user's presence at home, work, gym, shop, neighborhood, town, country, local weather conditions, etc; (c) trend-based statistics such as how things change over time; and (d) statistics from social networking contacts or peers, people in the same age group, and the like.

In other embodiments of the invention, users can indicate the “interest value” of a particular item of feedback, e.g., how interesting or useful they found this particular item of feedback or kind of feedback. This feedback can be used both to influence the feedback provided to that particular user as well as, e.g., being shared with an external service that can then be used to influence the feedback provided to other, comparable users (e.g., having similar ages, genders, interests, etc.). In one embodiment, this interest value may be computed using the following formula:

Interest Value (feedback)=function (Statistical Significance (feedback), . . . Matching Time Periods (feedback), Dissimilarity with Similar Users (feedback), . . . Likes of Feedback by Similar Users (feedback)).

The parameters of the Interest Value function can be set to predetermined values and subsequently adjusted according to the feedback received from the users. In operation, individual items of feedback can be processed by the Interest Value function prior to presentation to a user and presented if they exceed some threshold value, e.g.:

If (Interest Value (feedback)>threshold) Then present “feedback” to user

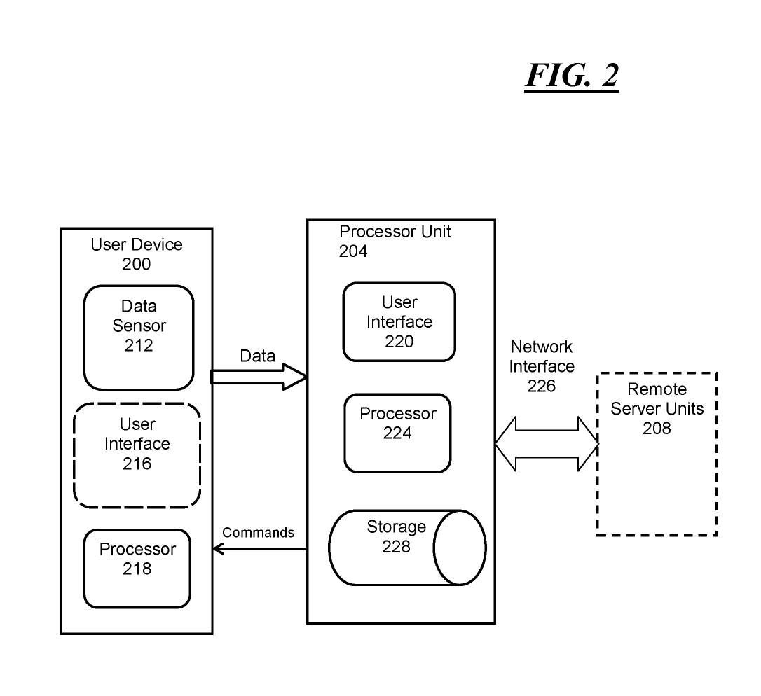

FIG. 2 is a block diagram illustrating one embodiment of a system for managing health habits, including a user device 200, an optional processor unit 204, and optional remote server units 208.

The user device 200 is primarily responsible for collecting the user's sensor data, although it also typically includes an interface 216 for collecting information from and/or providing information to the user. In some embodiments the user device 200 interoperates with a processor unit 204 that is primarily responsible for analyzing the collected data to determine parameters describing the physical fitness of the user.

The user device 200 can take a variety of forms, such as a smartwatch, bracelet, pendant, or any other type of wearable device, or an app running on a smartphone, etc. The processor unit 204 can likewise take a variety of forms, such as a server computer, desktop computer, laptop computer, tablet, phablet, smartphone, etc.

In some embodiments the user device 200 and the processor unit 204 are controlled by different users. For example, the user device 200 may be a smartwatch worn by a user while the processor unit 204 may be a desktop computer operated by another user, such as a nurse or a physician. In other embodiments, the functionality of the user device 200 and the processor unit 204 are offered by the same device. In still other embodiments, the processing capabilities of the processor unit 204 may be implemented across the processor unit 204 and one or more additional computing devices, such as remote server units 208. The following discussion assumes the user device 200 and the processor unit 204 to be separate physical devices for convenience, although this should not be construed to be limiting as to the overall scope of the present invention.

As illustrated, the user device 200 includes at least one sensor 212, an optional user interface 216, and a processor 218. The sensor 212 can comprise, for example, one or more of an accelerometer, an audio sensor, a video sensor, a location sensor, a movement sensor, an orientation sensor, a skin conductance sensor, a respiration sensor, a glucose level sensor, or a heart rate sensor. The user interface 216 can take many forms, but is typically appropriate to the form of the user device 200. Typical user interfaces 216 include a speech generator, a display (LCD, LED, CRT, E-Ink, etc.), a projector, a keyboard (physical or virtual), a speech recognition system, a touchscreen, etc. The processor 218 may be, e.g., an ARM-based or x86-based general purpose processing unit.

The optional processor unit 204 includes a user interface 220, a processor 224, a network interface 226, and a storage unit 228 which acts as a repository for the computer executable instructions that execute on the processor 224 and thereby provide the functionality for the present invention. The interface 220 may, like the interface 216, take a variety of forms that is appropriate to the particular form of the processor unit 204.

In operation, commands are sent from the processor unit 204 to the user device 200. Data is received by the processor unit 204 from the user device 200. Data may be transmitted to and received from a remote server unit 208 by the processor unit 204 via a network interface 226 in embodiments utilizing such remote server units 208.

The user may register a health habit via the user interface 216 on the user device 200, although the user may also register it using another device, such as processor unit 204. The sensor data is received by the processor 218 or the processor 224, processed to match previously registered health habit data packets and used to provide feedback to the user. The processor 218, 224 may also receive user data from other data sources, such as records of appointments, eating histories, contact information, driving directions, etc.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved.

Additionally, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

The description and illustration of one or more embodiments provided in this application are not intended to limit or restrict the scope of the present disclosure as claimed in any way. The embodiments, examples, and details provided in this application are considered sufficient to convey possession and enable others to make and use the best mode of the claimed embodiments. The claimed embodiments should not be construed as being limited to any embodiment, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the general inventive concept embodied in this application that do not depart from the broader scope of the claimed embodiments.

1. A method for managing a user's health habits with a computing unit, the method comprising:
receiving user data at said computing unit from at least one data source;
receiving a registration of a health habit at a user interface during or soon after performance of the health habit;
associating said user data with the health habit in response to the receipt of the registration of the health habit; and
providing context-appropriate feedback corresponding to recurrence of the health habit based on the association.

2. The method of claim 1, further comprising identifying a registered health habit from subsequently received user data by matching the subsequently received user data with the user data associated with the registered health habit.

3. The method of claim 2, wherein identifying a registered health habit comprises computing a cross-correlation between the subsequently received user data and the user data associated with the registered health habit.

4. The method of claim 2, wherein providing feedback to the user is based on the identified health habit.

5. The method of claim 4, wherein the feedback concerns at least one of the user data associated with the health habit, the subsequently received user data, and differences between the user data associated with the health habit and the subsequently received user data.

6. The method of claim 1, wherein the user data is selected from the group consisting of the user's calendar data, the user's communication data, the user's vital signs data, the user's motion data, the user's position data, the user's electronic transaction data, the user's height above sea level, the identity of specific persons nearby the user, and the user's weather data.

7. The method of claim 1, wherein the at least one data source is selected from the group consisting of an accelerometer, an audio sensor, a video sensor, a location sensor, a movement sensor, an orientation sensor, a skin conductance sensor, a respiration sensor, a glucose level sensor, and a heart rate sensor.

8. (canceled)

9. The method of claim 1, wherein the health habit is suggested by the computing unit prior to registration.

10. A system for managing a user's health habits, the system comprising:
a processor;
at least one data source; and
computer executable instructions operative on the processor for:
receiving user data from the at least one data source;
receiving a registration of a health habit at a user interface during or soon after performance of the health habit;
associating the user data with the health habit in response to the receipt of the registration of the health habit; and
providing context-appropriate feedback corresponding to recurrence of the health habit based on the association.

11. The system of claim 10, further comprising computer executable instructions operative on the processor for identifying a registered health habit from received user data by matching subsequently received user data with the user data associated with the registered health habit.

12. The system of claim 11, further comprising computer executable instructions operative on the processor for providing the feedback to the user based on the identified health habit.

13. The system of claim 10, wherein the user data is selected from the group consisting of the user's calendar data, the user's communication data, the user's vital signs data, the user's motion data, the user's position data, the user's electronic transaction data, and the user's weather data.

14. The system of claim 12, wherein the feedback concerns at least one of the user data associated with the health habit, the subsequently received user data, and differences between the user data associated with the health habit and the subsequently received user data.

15. The system of claim 11, wherein the computer executable instructions for identifying a registered health habit comprise computer executable instructions for computing a cross-correlation between the subsequently received user data and the user data associated with the registered health habit.

16. The system of claim 10, wherein the at least one data source is selected from the group consisting of an accelerometer, an audio sensor, a video sensor, a location sensor, a movement sensor, an orientation sensor, and a heart rate sensor.

17. (canceled)

18. The system of claim 10, further comprising computer executable instructions operative on the processor for suggesting the health habit that is subsequently registered.

19. A non-transitory computer readable storage medium comprising executable code that, when executed by a processor causes the processor to:
receive user data from at least one data source;
receive a registration of a health habit at a user interface during or soon after performance of the health habit;
associate the user data with the health habit in response to the receipt of the registration of the health habit; and
provide context-appropriate feedback corresponding to recurrence of the health habit based on the association.

20. The non-transitory computer readable storage medium of claim 19, wherein the executable code, when executed by the processor, causes the processor to match subsequently received user data with the user data associated with the registered health habit.

21. The non-transitory computer readable storage medium of claim 20, wherein the executable code, when executed by the processor, causes the processor to provide feedback to the user based on the identified health habit.

22. The non-transitory computer readable storage medium of claim 19, wherein the user data is selected from the group consisting of the user's calendar data, the user's communication data, the user's vital signs data, the user's motion data, the user's position data, the user's electronic transaction data, and the user's weather data.

23. The non-transitory computer readable storage medium of claim 21, wherein the feedback concerns at least one of the user data associated with the health habit, the subsequently received user data, and differences between the user data associated with the health habit and the subsequently received user data.

24. The non-transitory computer readable storage medium of claim 20, wherein the executable code, when executed by the processor, causes the processor to compute a cross-correlation between the subsequently received user data and the user data associated with the registered health habit.

25. (canceled)

26. The non-transitory computer readable storage medium of claim 19, wherein the executable code, when executed by the processor, causes the processor to suggest the health habit that is subsequently registered.
